(19) United States
(12) Patent Application Publication
(10) Pub. No.: US 2009/0123599 A1
(43) Pub. Date: May 14, 2009

(54) Cysteine Synthase Gene and Use Thereof

(75) Inventor: Yoshihiro Nakao, Osaka (JP)

Correspondence Address: DRINKER BIDDLE & REATH (DC), 1500 K STREET, N.W., SUITE 1100, WASHINGTON, DC, 20005-1209, US

(73) Assignee: SUNTORY LIMITED, Osaka (JP)

(21) Appl. No.: 11/886,790

(22) PCT Filed: Aug. 21, 2006
(86) PCT No.: PCT/JP2006/316785

§ 371 Date: Sep. 20, 2007

(30) Foreign Application Priority Data
Aug. 22, 2005 (JP) 2005-240350
Feb. 23, 2006 (JP) 2006-047554

(51) Int. Cl. C12N 9/00; C12N 15/52; C12N 15/81; C12N 1/19; C12C 11/00; C12G 1/022; C12H 1/00; C12G 3/02

(52) U.S. Cl. 426/15; 536/23.2; 435/183; 435/320.1; 435/254.2; 426/11; 426/16; 426/592

(57) ABSTRACT

The present invention relates to a brewery yeast having controlled hydrogen sulfide-producing capability, a process for producing alcoholic beverages with controlled hydrogen sulfide amount. More particularly, the present invention relates to a yeast whose hydrogen sulfide-producing capability that increases the product flavor is controlled by enhancing the expression level of YGR012W gene encoding brewery yeast cysteine synthase Ygr012wp, particularly non-ScYGR012W gene specific to lager brewing yeast, and to a method for producing alcoholic beverages with said yeast.

### TECHNICAL FIELD

The present invention relates to a cysteine synthase gene and to uses of the gene. The invention relates in particular to a brewer's yeast which produces alcoholic beverages of excellent flavor, alcoholic beverages produced using such a yeast, and a method of producing such alcoholic beverages. More specifically, the invention relates to YGR012W gene which codes for the cysteine synthase Ygr012wp in brewer's yeast, particularly to a yeast which improves the flavor of product by increasing the level of expression of the non-ScYGR012W gene characteristic to beer yeast or ScYGR012W gene and to a method of producing alcoholic beverages using such a yeast.

### BACKGROUND ART

The beer yeast used in the production of commercial Pilsner-type light-colored beers has the property of forming hydrogen sulfide during the primary fermentation step. This hydrogen sulfide is one cause of the immature beer aroma that is undesirable for beer quality. To reduce this aroma below a threshold level, extension of secondary fermentation period or extension of maturation period is carried out.

Research on the factors affecting the formation of hydrogen sulfide, (Jangaard, N. O., Gress, H. S, and Coe, R. W.: Amer. Soc. Brew. Chem. Proc., p. 46 (1973); Kuroiwa, Y. and Hashimoto, N.: Brew. Dig, 45, 44 (1970); Hysert, D. W. and Morrison, N. M.: J. Amer. Soc. Brew. Chem., 34, 25 (1976)), and research on the development of a low hydrogen sulfide producing yeast using a mutation process or a cell fusion process (Nolzahm, S. W.: J. Amer. Soc. Brew. Chem., 35, 54 (1977)) for lowering the hydrogen sulfide level in beer have been reported.

All of these methods not only reduces the amount of hydrogen sulfide produced by yeast but also affects the other brewing properties of the yeast (fermentation rate, beer flavor). Hence, such a yeast that is well-suited for brewing beer has not been achieved yet. Recently, development of brewer's yeasts using genetic engineering technology has been carried out. Japanese Patent Application Laid-open No. H5-244955 discloses that a beer yeast in which a DNA fragment coding for cystathionine β-synthase has been inserted reduces the production of hydrogen sulfide. However, the degree of reduction was small. That is to say, the amount of hydrogen sulfide produced by the transformant was about 60 to 80% of that produced by the parent strain.

In yeast metabolism, hydrogen sulfide is produced in the process of reducing sulfate ions (SO₄²⁻) taken up from the medium. This metabolic system is a pathway for the biosynthesis of sulfur-containing amino acids such as methionine and cysteine. Detailed studies have been published on the enzyme engaged in each stage of the pathway and its gene (MET17 gene) (see Tabor, H. and Tabor, C. W., eds., Methods in Enzymology, Vol. 17B (London: Academic Press, 1971); and Jakoby, W. B. and Griffith, O. W., eds., Methods in Enymology, Vol. 143 (London: Academic Press, 1987)).

O-acetylhomoserinesulfhydrorelace is an enzyme which transfers a sulfur atom from hydrogen sulfide to O-acetylhomoserine, and is encoded by the MET17 gene. This enzyme also transfers a sulfur atom to O-acetylserine. It has been reported that a beer yeast strain which the MET17 gene from Saccharomyces cerevisiae X2180-1A has been constitutively expressed produces reduced amount of hydrogen sulfide, which is about 2% of the level in the parent strain (Japanese Patent Application Laid-open No. H7-303475).

Further, cysteine synthase is a enzyme which transfers a sulfur atom from a hydrogen sulfide to O-acetyl-L-serine. A gene of the enzyme have not been identified in Saccharomyces cerevisiae, although it has been identified in other microorganisms.

Recently, a number of genome sequences were determined. Identification of a gene and estimation of a function of the gene by comparison with known genes were performed based on the determined genome sequences. Further, orthologous genes (i.e., a pair of genes of two different species, wherein the genes are originated from the same gene of a shared ancestry, and current function of the genes are the same.) were estimated by comparison analysis of a numerous genes present in numerous genome sequences of organisms. A gene encoding a cysteine synthase in Saccharomyces cerevisiae is estimated to be YGR012W by orthologue analysis of microorganism genome (R. L. Tatusov., M. Y. Galperin., D. A. Natale., E. V. Koonin; Nucleoc. Acids. Research., 28, 33, 2000). However, a function of the gene has not been confirmed experimentally. Further, it has not been determined if the gene is related to the production amount of hydrogen sulfide.

### DISCLOSURE OF INVENTION

As noted above, variant strains have been developed in order to lower the amount of hydrogen sulfide produced in the final product. As a result, unexpected delays in fermentation and increases in undesirable flavor components was observed in some cases, which makes the practical use of such yeasts questionable. There were thus demands for a method of developing yeasts which produces less hydrogen sulfide without compromising either the fermentation rate or the product quality.

The present inventors made exhaustive studies to solve the above problems, and as a result succeeded in identifying and isolating a gene encoding a cysteine synthase from lager brewing yeast. Moreover, a yeast in which the obtained gene was transformed and expressed was produced to confirm reduction of the amount of hydrogen sulfide production, thereby completing the present invention.

Thus, the present invention relates to a novel cysteine synthase gene existing in a lager brewing yeast, to a protein encoded by said gene, to a transformed yeast in which the expression of said gene is controlled, to a method for controlling the amount of hydrogen sulfide production in a product by using a yeast in which the expression of said gene is controlled. More specifically, the present invention provides the following polynucleotides, a vector comprising said polynucleotide, a transformed yeast introduced with said vector, a method for producing alcoholic beverages by using said transformed yeast, and the like.

(1) A polynucleotide selected from the group consisting of:

(a) a polynucleotide comprising a polynucleotide consisting of the nucleotide sequence of SEQ ID NO:1;

(b) a polynucleotide comprising a polynucleotide encoding a protein consisting of the amino acid sequence of SEQ ID NO:2;

(c) a polynucleotide comprising a polynucleotide encoding a protein consisting of the amino acid sequence of SEQ ID NO:2 with one or more amino acids thereof being deleted, substituted, inserted and/or added, and having a cysteine synthase activity;

(d) a polynucleotide comprising a polynucleotide encoding a protein having an amino acid sequence having 60% or higher identity with the amino acid sequence of SEQ ID NO:2, and having a cysteine synthase activity;

(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having a cysteine synthase activity; and

(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO:2 under stringent conditions, and which encodes a protein having a cysteine synthase activity.

(2) The polynucleotide of (1) above selected from the group consisting of:

(g) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has a cysteine synthase activity;

(h) a polynucleotide encoding a protein having 90% or higher identity with the amino acid sequence of SEQ ID NO: 2, and having a cysteine synthase activity; and

(i) a polynucleotide which hybridizes to SEQ ID NO: 1 or which hybridizes to a nucleotide sequence complementary to the nucleotide sequence of SEQ ID NO: 1 under high stringent conditions, and which encodes a protein having a cysteine synthase activity.

(3) The polynucleotide of (1) above comprising a polynucleotide consisting of SEQ ID NO: 1.

(4) The polynucleotide of (1) above comprising a polynucleotide encoding a protein consisting of SEQ ID NO: 2.

(5) The polynucleotide of any one of (1) to (4) above, wherein the polynucleotide is DNA.

(6) A protein encoded by the polynucleotide of any one of (1) to (5) above.

(7) A vector comprising the polynucleotide of any one of (1) to (5) above.

(7a) The vector of (7) above, which comprises the expression cassette comprising the following components:

(x) a promoter that can be transcribed in a yeast cell;

(y) any of the polynucleotides described in (1) to (5) above linked to the promoter in a sense or antisense direction; and

(z) a signal that can function in a yeast with respect to transcription termination and polyadenylation of a RNA molecule.

(8) A vector comprising the polynucleotide selected from the group consisting of:

(j) a polynucleotide encoding a protein consisting of the amino acid sequence of SEQ ID NO: 6, or encoding an amino acid sequence of SEQ ID NO: 6 wherein 1 to 10 amino acids thereof is deleted, substituted, inserted, and/or added, and wherein said protein has a cysteine synthase activity;

(k) a polynucleotide encoding a protein having 90% or higher identity with the amino acid sequence of SEQ ID NO: 6, and having a cysteine synthase activity; and

(l) a polynucleotide which hybridizes to SEQ ID NO: 5 or which hybridizes to a nucleotide sequence complementary to the nucleotide sequence of SEQ ID NO: 5 under high stringent conditions, and which encodes a protein having a cysteine synthase activity.

(9) A yeast, wherein the vector of (7) or (8) above is introduced.

(10) The yeast of (9) above, wherein hydrogen sulfide-producing ability is reduced by introducing the vector of (7) or (8) above.

(11) The yeast of (10) above, wherein a hydrogen sulfide-producing ability is reduced by increasing an expression level of the protein of (6) above.

(12) A method for producing an alcoholic beverage by using the yeast of any one of (9) to (11) above.

(13) The method for producing an alcoholic beverage of (12) above, wherein the brew is a malt beverage.

(14) The method for producing an alcoholic beverage of (12) above, wherein the brew is a wine.

(15) An alcoholic beverage, which is produced by the method of any one of (12) to (14) above.

(16) A method for assessing a test yeast for its hydrogen sulfide-producing ability, comprising using a primer or a probe designed based on a nucleotide sequence of a cysteine synthase gene having the nucleotide sequence of SEQ ID NO: 1 or SEQ ID NO: 5.

(16a) A method for selecting a yeast having a low hydrogen sulfide-producing ability by using the method in (16) above.

(16b) A method for producing an alcoholic beverage (for example, beer) by using the yeast selected with the method in (16a) above.

(17) A method for assessing a test yeast for its hydrogen sulfide-producing capability, comprising: culturing a test yeast; and measuring an expression level of a cysteine synthase gene having the nucleotide sequence of SEQ ID NO: 1 or SEQ ID NO: 5.

(17a) A method for selecting a yeast having a low hydrogen sulfide-producing capability, which comprises assessing a test yeast by the method described in (17) above and selecting a yeast having a high expression level of the cysteine synthase gene.

(17b) A method for producing an alcoholic beverage (for example, beer) by using the yeast selected with the method in (17a) above.

(18) A method for selecting a yeast, comprising: culturing test yeasts; quantifying the protein of (6) above or measuring an expression level of a cysteine synthase gene having the nucleotide sequence of SEQ ID NO: 1 or SEQ ID NO: 5; and selecting a test yeast having said protein amount or said gene expression level according to a target capability of producing hydrogen sulfide.

(18a) A method for selecting a yeast, comprising: culturing test yeasts; measuring a hydrogen sulfide-producing capability or a cysteine synthase activity; and selecting a test yeast having a target capability of producing hydrogen sulfide or a target cysteine synthase activity.

(19) The method for selecting a yeast of (18) above, comprising: culturing a reference yeast and test yeasts; measuring an expression level of a cysteine synthase gene having the nucleotide sequence of SEQ ID NO: 1 or SEQ ID NO: 5 in each yeast; and selecting a test yeast having the gene expressed higher than that in the reference yeast.

(20) The method for selecting a yeast of (18) above comprising: culturing a reference yeast and test yeasts; quantifying the protein of (6) above in each yeast; and selecting a test yeast having said protein for a larger amount than that in the reference yeast. That is, the method for selecting a yeast of (18) above comprising: culturing plural yeasts; quantifying the protein of (6) above in each yeast; and selecting a test yeast having a large amount of the protein from them.

(21) A method for producing an alcoholic beverage comprising: conducting fermentation for producing an alcoholic beverage using the yeast according to any one of (9) to (11) or a yeast selected by the method according to any one of (18) to (20); and adjusting the production amount of hydrogen sulfide.

### BRIEF DESCRIPTION OF DRAWINGS

FIG. 1 shows the cell growth with time upon beer brewing testing. The horizontal axis represents fermentation time while the vertical axis represents optical density at 660 nm (OD660).

FIG. 2 shows the extract consumption with time upon test brew of beer. The horizontal axis represents fermentation time while the vertical axis represents apparent extract concentration (w/w %)

FIG. 3 shows the expression behavior of non-ScYGR012W gene in yeasts upon test brew of beer. The horizontal axis represents fermentation time while the vertical axis represents the intensity of detected signal.

FIG. 4 shows the cell growth with time upon test brew of beer using parent strain and non-ScYGR012W highly expressed strain. The horizontal axis represents fermentation time while the vertical axis represents optical density at 660 nm (OD660).

FIG. 5 shows the sugar consumption with time upon test brew of beer using parent strain and non-ScYGR012W highly expressed strain. The horizontal axis represents fermentation time while the vertical axis represents apparent extract concentration (w/w %).

FIG. 6 shows the cell growth with time upon test brew of beer. The horizontal axis represents fermentation time while the vertical axis represents optical density at 660 nm (OD660).

FIG. 7 shows the extract consumption with time upon test brew of beer. The horizontal axis represents fermentation time while the vertical axis represents apparent extract concentration (w/w %).

FIG. 8 shows the expression behavior of ScYGR012W gene in yeasts upon test brew of beer. The horizontal axis represents fermentation time while the vertical axis represents the intensity of detected signal.

FIG. 9 shows the cell growth with time upon test brew of beer using parent strain and ScYGR012W highly expressed strain. The horizontal axis represents fermentation time while the vertical axis represents optical density at 660 nm (OD660).

FIG. 10 shows the extract consumption with time upon test brew of beer using parent strain and ScYGR012W highly expressed strain. The horizontal axis represents fermentation time while the vertical axis represents apparent extract concentration (w/w %).

### BEST MODES FOR CARRYING OUT THE INVENTION

The present inventors conceived that it is possible to lower hydrogen sulfide effectively by increasing a cysteine synthase activity of the yeast. The present inventors have studied based on this conception and as a result, isolated and identified non-ScYGR012W gene encoding a cysteine synthase unique to lager brewing yeast based on the lager brewing yeast genome information mapped according to the method disclosed in Japanese Patent Application Laid-Open No. 2004-283169. The nucleotide sequence of the gene is represented by SEQ ID NO: 1. Further, an amino acid sequence of a protein encoded by the gene is represented by SEQ ID NO: 2. In addition, the present inventors have isolated and identified ScYGR012W gene encoding a cysteine synthase of lager brewing yeast. The nucleotide sequence of the gene is represented by SEQ ID NO: 5. Further, an amino acid sequence of a protein encoded by the gene is represented by SEQ ID NO: 6.

### 1. Polynucleotide of the Invention

First of all, the present invention provides (a) a polynucleotide comprising a polynucleotide of the nucleotide sequence of SEQ ID NO: 1 or SEQ ID NO: 5; and (b) a polynucleotide comprising a polynucleotide encoding a protein of the amino acid sequence of SEQ ID NO:2 or SEQ ID NO: 6. The polynucleotide can be DNA or RNA.

The target polynucleotide of the present invention is not limited to the polynucleotide encoding a cysteine synthase gene derived from lager brewing yeast and may include other polynucleotides encoding proteins having equivalent functions to said protein. Proteins with equivalent functions include, for example, (c) a protein of an amino acid sequence of SEQ ID NO: 2 or SEQ ID NO: 6 with one or more amino acids thereof being deleted, substituted, inserted and/or added and having cysteine synthase activity.

Such proteins include a protein consisting of an amino acid sequence of SEQ ID NO: 2 or SEQ ID NO: 6 with, for example, 1 to 100, 1 to 90, 1 to 8, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having a cysteine synthase activity. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher identity with the amino acid sequence of SEQ ID NO: 2 or SEQ ID NO: 6, and having a cysteine synthase activity. In general, the percentage identity is preferably higher.

Cysteine synthase activity may be measured, for example, by a method of Thomas et al. as described in J. Biol. Chem. 45: 28187-28192 (1994).

Furthermore, the present invention also contemplates (e) a polynucleotide comprising a polynucleotide which hybridizes to a polynucleotide consisting of a nucleotide sequence complementary to the nucleotide sequence of SEQ ID NO: 1 or SEQ ID NO: 5 under stringent conditions and which encodes a protein having cysteine synthase activity; and (f) a polynucleotide comprising a polynucleotide which hybridizes to a polynucleotide complementary to a nucleotide sequence of encoding a protein of SEQ ID NO: 2 or SEQ ID NO: 6 under stringent conditions, and which encodes a protein having cysteine synthase activity.

Herein, “a polynucleotide that hybridizes under stringent conditions” refers to a polynucleotide, such as a DNA, obtained by a colony hybridization technique, a plaque hybridization technique, a southern hybridization technique or the like using all or part of polynucleotide of a nucleotide sequence complementary to the nucleotide sequence of SEQ ID NO: 1 or SEQ ID NO: 5, or polynucleotide encoding the amino acid sequence of SEQ ID NO: 2 or SEQ ID NO: 6 as a probe. The hybridization method may be a method described, for example, in MOLECULAR CLONING 3rd Ed., CURRENT PROTOCOLS IN MOLECULAR BIOLOGY, John Wiley & Sons 1987-1997.

The term “stringent conditions” as used herein may be any of low stringency conditions, moderate stringency conditions or high stringency conditions. “Low stringency conditions” are, for example, 5×SSC, 5×Denhardt's solution, 0.5% SDS, 50% formamide at 32° C. “Moderate stringency conditions” are, for example, 5×SSC, 5×Denhardt's solution, 0.5% SDS, 50% formamide at 42° C. “High stringency conditions” are, for example, 5×SSC, 5×Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotides encoding the amino acid sequence of SEQ ID NO: 2 or SEQ ID NO: 6 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul S F et al., J. Mol. Biol. 215: 403, 1990). When a nucleotide sequence is sequenced using BLASTN, the parameters are, for example, score=100 and word length=12. When an amino acid sequence is sequenced using BLASTX, the parameters are, for example, score=50 and word length=3. When BLAST and Gapped BLAST programs are used, default parameters for each of the programs are employed.

### 2. Protein of the Present Invention

The present invention also provides proteins encoded by any of the polynucleotides (a) to (1) above. A preferred protein of the present invention comprises an amino acid sequence of SEQ ID NO:2 or SEQ ID NO: 6 with one or several amino acids thereof being deleted, substituted, inserted and/or added, and has a cysteine synthase activity.

Such protein includes those having an amino acid sequence of SEQ ID NO: 2 or SEQ ID NO: 6 with amino acid residues thereof of the number mentioned above being deleted, substituted, inserted and/or added and having a cysteine synthase activity. In addition, such protein includes those having homology as described above with the amino acid sequence of SEQ ID NO: 2 or SEQ ID NO: 6 and having a cysteine synthase activity.

Such proteins may be obtained by employing site-directed mutation described, for example, in MOLECULAR CLONING 3rd Ed., CURRENT PROTOCOLS IN MOLECULAR BIOLOGY, Nuc. Acids. Res., 10: 6487 (1982), Proc. Natl. Acad. Sci. USA 79: 6409 (1982), Gene 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminopropionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenylmethyloxycarbonyl method) and tBoc method (t-butyloxycarbonyl method). In addition, peptide synthesizers available from, for example, Advanced ChemTech, PerkinElmer, Pharmacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides (for example, DNA) described in (a) to (1) above. Generally, the vector of the present invention comprises an expression cassette including as components (x) a promoter that can transcribe in a yeast cell; (y) a polynucleotide (for example, DNA) described in any of (a) to (l) above that is linked to the promoter in sense or antisense direction; and (z) a signal that functions in the yeast with respect to transcription termination and polyadenylation of RNA molecule. According to the present invention, in order to highly express the protein of the invention described above upon brewing alcoholic beverages (e.g., beer) described below, these polynucleotides are introduced into the promoter in the sense direction to promote expression of the polynucleotide (for example, DNA) described in any of (a) to (i) above.

A vector introduced in the yeast may be any of a multicopy type (YEp type), a single copy type (YCp type), or a chromosome integration type (YIp type). For example, YEp24 (J. R. Broach et al., EXPERIMENTAL MANIPULATION OF GENE EXPRESSION, Academic Press, New York, 83, 1983) is known as a YEp type vector, YCp50 (M. D. Rose et al., Gene 60: 237, 1987) is known as a YCp type vector, and Yip5 (K. Struhl et al., Proc. Natl. Acad. Sci. USA, 76: 1035, 1979) is known as a Yip type vector, all of which are readily available.

Promoters/terminators for adjusting gene expression in yeast may be in any combination as long as they function in the brewery yeast and they are not influenced by constituents in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail, for example, in M. F. Tuite et al., EMBO J., 1, 603 (1982), and are readily available by known methods.

Since an auxotrophy marker cannot be used as a selective marker upon transformation for a brewery yeast, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast that can be used for brewing, for example, brewery yeasts for beer, wine and sake. Specifically, yeasts such as genus Saccharomyces may be used. According to the present invention, a lager brewing yeast, for example, Saccharomyces pastorianus W34/70, Saccharomyces carlsbergensis NCYC453 or NCYC456, or Saccharomyces cerevisiae NBRC1951, NBRC1952, NBRC1953 or NBRC1954 may be used. In addition, whisky yeasts such as Saccharomyces cerevisiae NCYC90, wine yeasts such as wine yeasts #1, 3 and 4 from the Brewing Society of Japan, and sake yeasts such as sake yeast #7 and 9 from the Brewing Society of Japan may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method (Proc. Natl. Acad. Sci. USA, 75: 1929 (1978)), a lithium acetate method (J. Bacteriology, 153: 163 (1983)), and methods described in Proc. Natl. Acad. Sci. USA, 75: 1929 (1978), METHODS IN YEAST GENETICS, 2000 Edition: A Cold Spring Harbor Laboratory Course Manual.

More specifically, a host yeast is cultured in a standard yeast nutrition medium (e.g., YEPD medium (Genetic Engineering. Vol. 1, Plenum Press, New York, 117 (1979)), etc.) such that OD600 nm will be 1 to 6. This culture yeast is collected by centrifugation, washed and pre-treated with alkali ion metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in MOLECULAR CLONING 3rd Ed., and METHODS IN YEAST GENETICS, A LABORATORY MANUAL (Cold Spring Harbor Laboratory Press, Cold Spring Harbor, N.Y.).

### 4. Method of Producing Alcoholic Beverages According to the Present Invention and Alcoholic Beverages Produced by the Method

The vector of the present invention described above is introduced into a yeast suitable for brewing a target alcoholic product. This yeast can be used to produce a desired alcoholic beverage with enhanced flavor with a lowered content of hydrogen sulfide. In addition, yeasts to be selected by the yeast assessment method of the present invention described below can also be used. The target alcoholic beverages include, for example, but not limited to beer, sparkling liquor (happoushu) such as a beer-taste beverage, wine, whisky, sake and the like.

In order to produce these alcoholic beverages, a known technique can be used except that a brewery yeast obtained according to the present invention is used in the place of a parent strain. Since materials, manufacturing equipment, manufacturing control and the like may be exactly the same as the conventional ones, there is no need of increasing the cost for producing alcoholic beverages with a lowered content of hydrogen sulfide. Thus, according to the present invention, alcoholic beverages with enhanced flavor can be produced using the existing facility without increasing the cost.

### 5. Yeast Assessment Method of the Invention

The present invention relates to a method for assessing a test yeast for its hydrogen sulfide-producing capability by using a primer or a probe designed based on a nucleotide sequence of a cysteine synthase gene having the nucleotide sequence of SEQ ID NO:1 or SEQ ID NO: 5. General techniques for such assessment method is known and is described in, for example, WO01/040514, Japanese Laid-Open Patent Application No. 8-205900 or the like. This assessment method is described in below.

First, genome of a test yeast is prepared. For this preparation, any known method such as Hereford method or potassium acetate method may be used (e.g., METHODS IN YEAST GENETICS, Cold Spring Harbor Laboratory Press, 130 (1990)). Using a primer or a probe designed based on a nucleotide sequence (preferably, ORF sequence) of the cysteine synthase gene, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of the capability of the yeast to produce hydrogen sulfide as determined by whether the molecular weight of the amplified product is a size that contains the DNA molecule of the specific part. In addition, by analyzing the nucleotide sequence of the amplified product, the capability may be predicted and/or assessed more precisely.

Moreover, in the present invention, a test yeast is cultured to measure an expression level of the cysteine synthase gene having the nucleotide sequence of SEQ ID NO: 1 or SEQ ID NO: 5 to assess the test yeast for its hydrogen sulfide-producing capability. In measuring an expression level of the cysteine synthase gene, the test yeast is cultured and then mRNA or a protein resulting from the cysteine synthase gene is quantified. The quantification of mRNA or protein may be carried out by employing a known technique. Messenger RNA (mRNA) may be quantified, by Northern hybridization or quantitative RT-PCR, while protein may be quantified, for example, by Western blotting (CURRENT PROTOCOLS IN MOLECULAR BIOLOGY, John Wiley & Sons 1994-2003).

Furthermore, test yeasts are cultured and expression levels of the cysteine synthase gene having the nucleotide sequence of SEQ ID NO: 1 or SEQ ID NO: 5 are measured to select a test yeast with the gene expression level according to the target capability of producing hydrogen sulfide, thereby selecting a yeast favorable for brewing desired alcoholic beverages. In addition, a reference yeast and a test yeast may be cultured so as to measure and compare the expression level of the gene in each of the yeasts, thereby selecting a favorable test yeast. More specifically, for example, a reference yeast and one or more test yeasts are cultured and an expression level of the cysteine synthase gene having the nucleotide sequence of SEQ ID NO: 1 or SEQ ID NO: 5 is measured in each yeast. By selecting a test yeast with the gene expressed higher than that in the reference yeast, a yeast suitable for brewing desired alcoholic beverages can be selected.

Alternatively, test yeasts are cultured and a yeast with a lower hydrogen sulfide-producing capability or with a higher or lower cysteine synthase activity is selected, thereby selecting a yeast suitable for brewing desired alcoholic beverages.

In these cases, the test yeasts or the reference yeast may be, for example, a yeast introduced with the vector of the invention, a yeast in which an expression of a polynucleotide (DNA) of the invention has been controlled, an artificially mutated yeast or a naturally mutated yeast. The production amount of hydrogen sulfide can be measured by, for example, any of the methods described in Brauwissenschaft. 31. 1 (1978), Applied Environm. Microbiol. 66: 4421-4426 (2000), or J. Am. Soc. Brew. Chem. 53: 58-62 (1995). cysteine synthase activity can be measured by, for example, a method described in J. Biol. Chem. 45: 28187-28192 (1994). The mutation treatment may employ any methods including, for example, physical methods such as ultraviolet irradiation and radiation irradiation, and chemical methods associated with treatments with drugs such as EMS (ethylmethane sulphonate) and N-methyl-N-nitrosoguanidine (see, e.g., Yasuji Oshima Ed., BIOCHEMISTRY EXPERIMENTS vol. 39, Yeast Molecular Genetic Experiments, pp. 67-75, JSSP).

In addition, examples of yeasts used as the reference yeast or the test yeasts include any yeasts that can be used for brewing, for example, brewery yeasts for beer, wine, sake and the like. More specifically, yeasts such as genus Saccharomyces may be used (e.g., S. pastorianus, S. cerevisiae, and S. carlsbergensis). According to the present invention, a lager brewing yeast, for example, Saccharomyces pastorianus W34/70; Saccharomyces carlsbergensis NCYC453 or NCYC456; or Saccharomyces cerevisiae NBRC1951, NBRC1952, NBRC1953 or NBRC1954 may be used. Further, wine yeasts such as wine yeasts #1, 3 and 4 from the Brewing Society of Japan; and sake yeasts such as sake yeast #7 and 9 from the Brewing Society of Japan may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

### Cloning of a Novel Cysteine Synthase (Non-ScYGR012W) Gene

A specific novel cysteine synthase gene non-ScYGR012W (SEQ ID NO: 1) of a lager brewing yeast were found as a result of a search utilizing the comparison database described in Japanese Patent Application Laid-Open No. 2004-283169. Based on the acquired nucleotide sequence information, primers non-ScYGR012W_for (SEQ ID NO: 3) and non-ScYGR012W_rv (SEQ ID NO: 4) were designed to amplify the full-length genes, respectively. PCR was carried out using chromosomal DNA of a genome sequencing strain, Saccharomyces pastorianus Weihenstephan 34/70 strain, also abbreviated to “W34/70 strain”, as a template to obtain DNA fragments (about 1.2 kb) including the full-length gene of non-ScYGR012W.

The thus-obtained non-ScYGR012W gene fragment was inserted into pCR2.1-TOPO vector (Invitrogen) by TA cloning. The nucleotide sequences of non-ScYGR012W gene were analyzed according to Sanger's method (F. Sanger, Science, 214: 1215, 1981) to confirm the nucleotide sequence.

### Example 2

### Analysis of Expression of Non-ScYGR012W Gene during Beer Fermentation

A fermentation test was conducted using a lager brewing yeast, Saccharomyces pastorianus W34/70 strain and then mRNA extracted from yeast cells during fermentation was detected by a DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

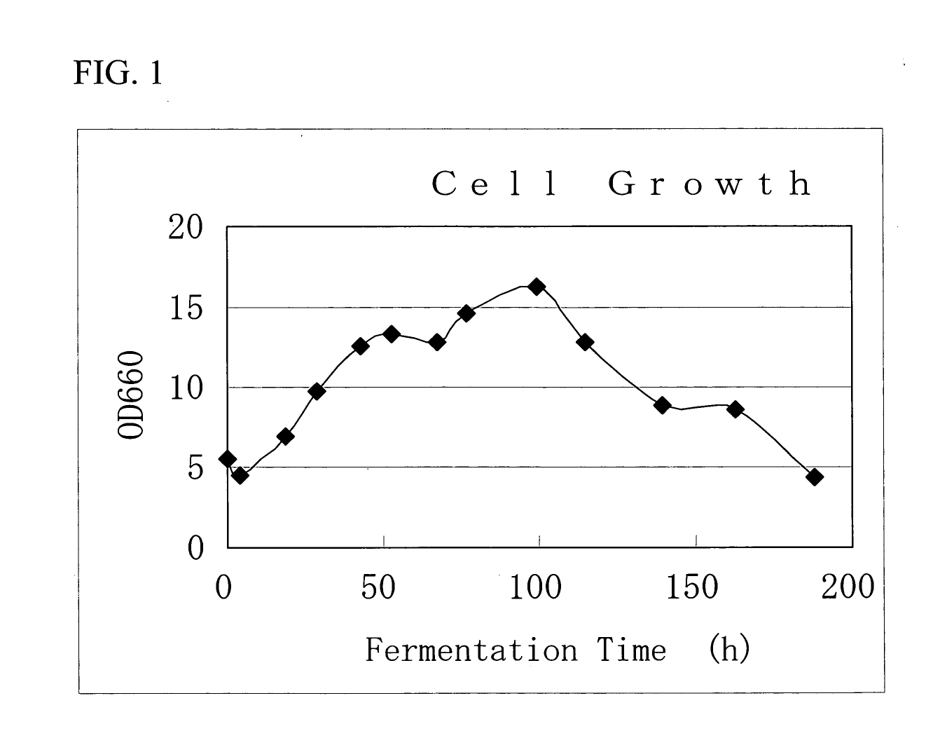

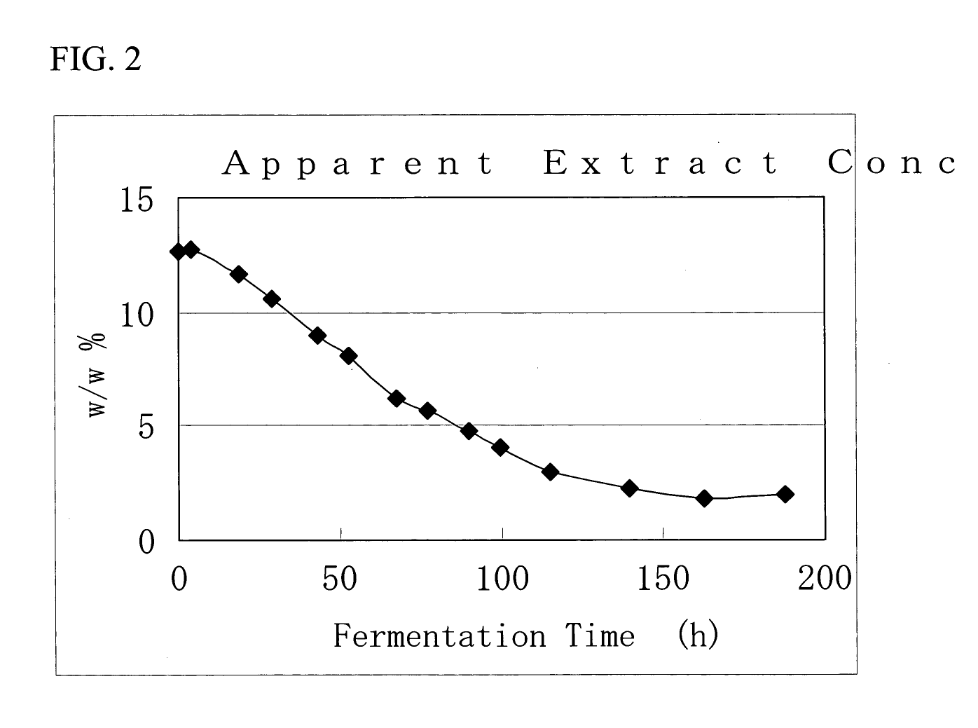

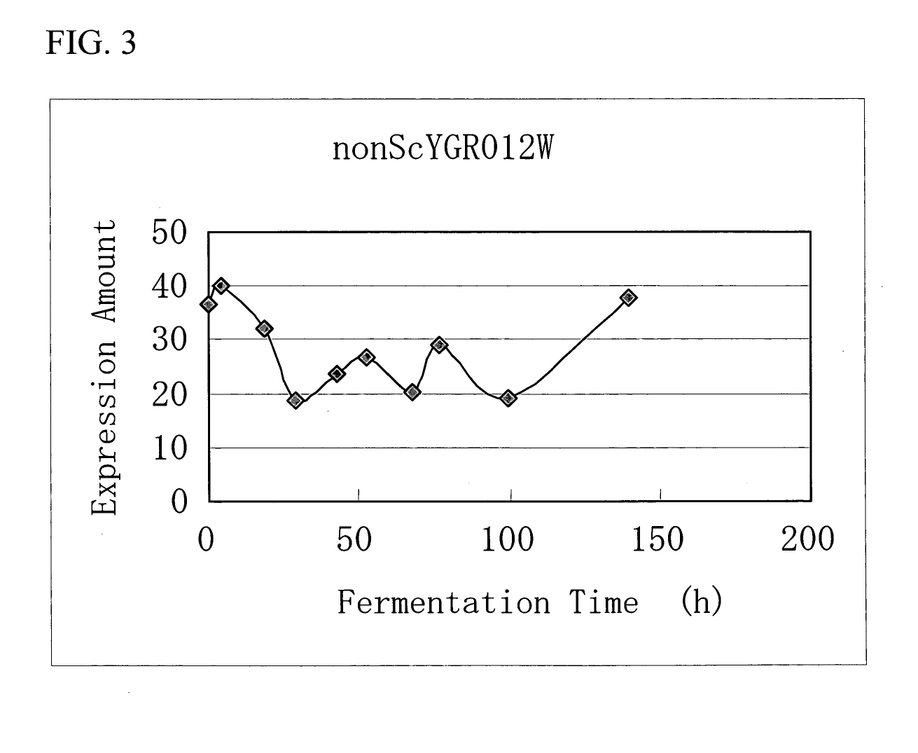

Sampling of fermented liquid was performed with time, and variation with time of yeast growth amount (FIG. 1) and apparent extract concentration (FIG. 2) was observed. Simultaneously, yeast cells were sampled to prepare mRNA, and the prepared mRNA was labeled with biotin and was hybridized to a beer yeast DNA microarray. The signal was detected using GCOS; GeneChip Operating Software 1.0 (manufactured by Affymetrix Co.). Expression pattern of non-ScYGR012W gene is shown in FIG. 3. As a result, it was confirmed that non-ScYGR012W gene was expressed in the general beer fermentation.

### Example 3

### High Expression of Non-ScYGR012W Gene

The non-ScYGR012W/pCR2.1-TOPO described in Example 1 was digested using the restriction enzymes SacI and NotI so as to prepare a DNA fragment containing the entire length of the protein-encoding region. This fragment was ligated to pYCGPYNot treated with the restriction enzymes SacI and NotI, thereby constructing the non-ScYGR012W high expression vector non-ScYGR012W/pYCGPYNot. pYCGPYNot is a YCp-type yeast expression vector. The inserted gene is highly expressed by the pyruvate kinase gene PYK1 promotor. The geneticin-resistant gene G418^{r} is included as the selection marker in the yeast, and the ampicillin-resistant gene Ampr is included as the selection marker in Escherichia coli.

Using the high expression vector prepared by the above method, the strain Saccharomyces pasteurianus Weihenstephaner 34/70 was transformed by the method described in Japanese Patent Application Laid-open No. H7-303475. The transformant was selected in a YPD plate culture (1% yeast extract, 2% polypeptone, 2% glucose, 2% agar) containing 300 mg/L of geneticin.

### Example 4

### Analysis of Amount of Hydrogen Sulfide Produced in Test Brew of Beer

A fermentation test was carried out under the following conditions using the parent strain (W34/70 strain) and the non-ScYGR012W highly expressed strain obtained in Example 3.

| | |
|---|---|
| Wort extract concentration | 12% |
| Wort content | 1 L |
| Wort dissolved oxygen concentration | approx. 8 ppm |
| Fermentation temperature | 15° C. (fixed) |
| Yeast pitching rate | 5 g wet yeast cells/L of wort |

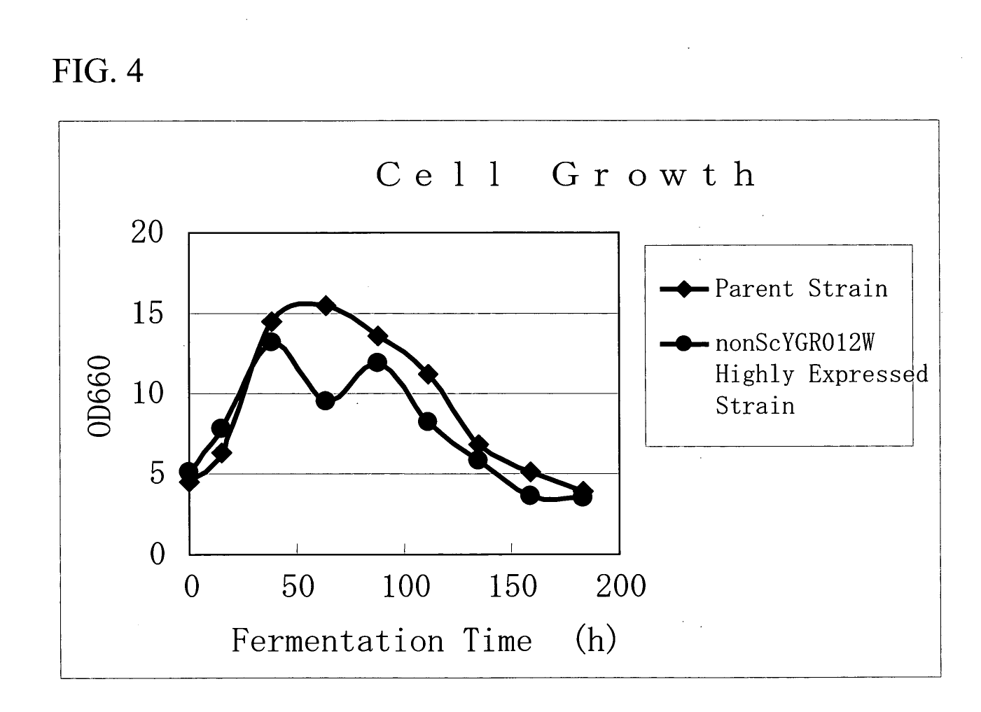

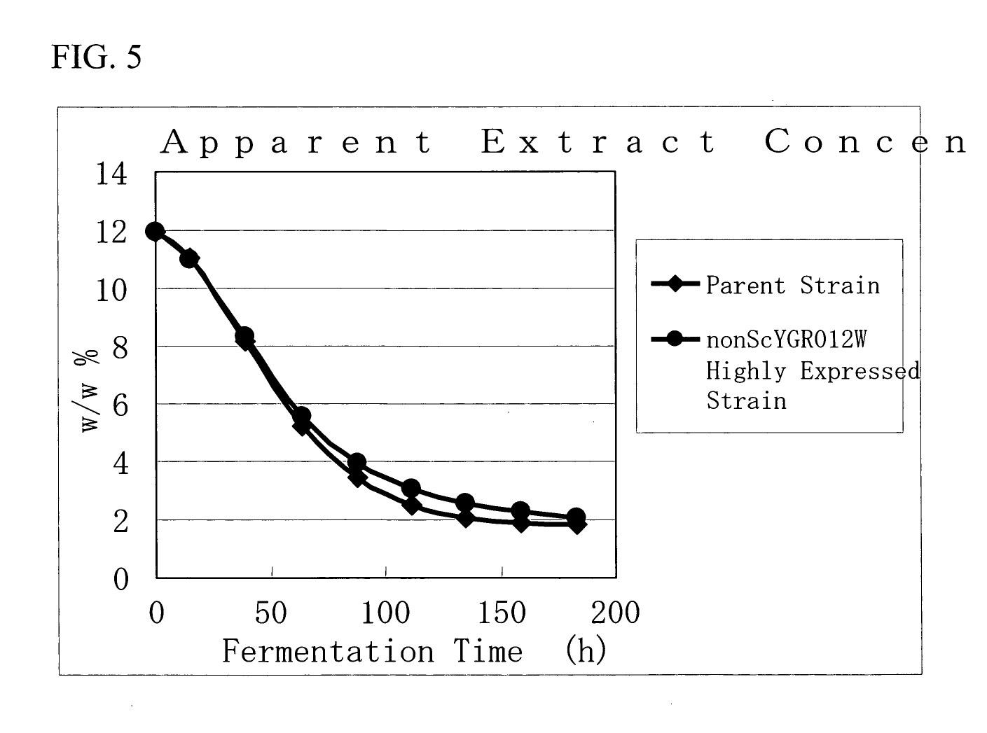

The fermentation broth was sampled over time, and the change over time in the yeast growth rate (OD660) (see FIG. 4) and the amount of extract consumed were determined (see FIG. 5). Quantitative determination of the hydrogen sulfide on completion of fermentation was carried out based on the method of Takahashi et al. (Brauwissenschaft 31, 1 (1978)). First, a sample containing a known concentration of hydrogen sulfide was measured and a standard curve for hydrogen sulfide was prepared from the peak area for the hydrogen sulfide detected. The amount of hydrogen sulfide was determined from the relationship between the standard curve and the area for the hydrogen sulfide detected in measurement of the fermentation broth under the same conditions as those used for analyzing the standard sample (Table 1).

TABLE 1
Amount of hydrogen sulfide in fermentation
broth at completion of fermentation.

| | Parent strain | non-ScYGR012W |
|---|---|---|
| | (W34/70 strain) | highly expressed strain |
| H₂S (ppb) | 22.1 | 3.3 |

The amount of hydrogen sulfide that had been produced on completion of fermentation was 22.1 ppb for the parent strain, whereas it was 3.3 ppb for the non-ScYGR012W highly expressed strain as described in Table 1. It was clear from these results that the amount of hydrogen sulfide production was reduced about 85% by high expression of the non-ScYGR012W gene.

### Example 5

### Cloning of a Cysteine Synthase (ScYGR012W) Gene

A cysteine synthase gene ScYGR012W (SEQ ID NO: 5) of a lager brewing yeast were found as a result of a search utilizing the comparison database described in Japanese Patent Application Laid-Open No. 2004-283169. Based on the acquired nucleotide sequence information, primers ScYGR012W_for (SEQ ID NO: 7) and ScYGR012W_rv (SEQ ID NO: 8) were designed to amplify the full-length genes, respectively. PCR was carried out using chromosomal DNA of a genome sequencing strain, Saccharomyces pastorianus Weihenstephan 34/70 strain, as a template to obtain DNA fragments (about 1.2 kb) including the full-length gene of ScYGR012W.

The thus-obtained ScYGR012W gene fragment was inserted into pCR2.1-TOPO vector (Invitrogen) by TA cloning. The nucleotide sequences of ScYGR012W gene were analyzed according to Sanger's method (F. Sanger, Science, 214: 1215, 1981) to confirm the nucleotide sequence.

### Example 6

### Analysis of Expression of ScYGR012W Gene During Beer Fermentation

A fermentation test was conducted using a lager brewing yeast, Saccharomyces pastorianus W34/70 strain and then mRNA extracted from yeast cells during fermentation was analyzed by a DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

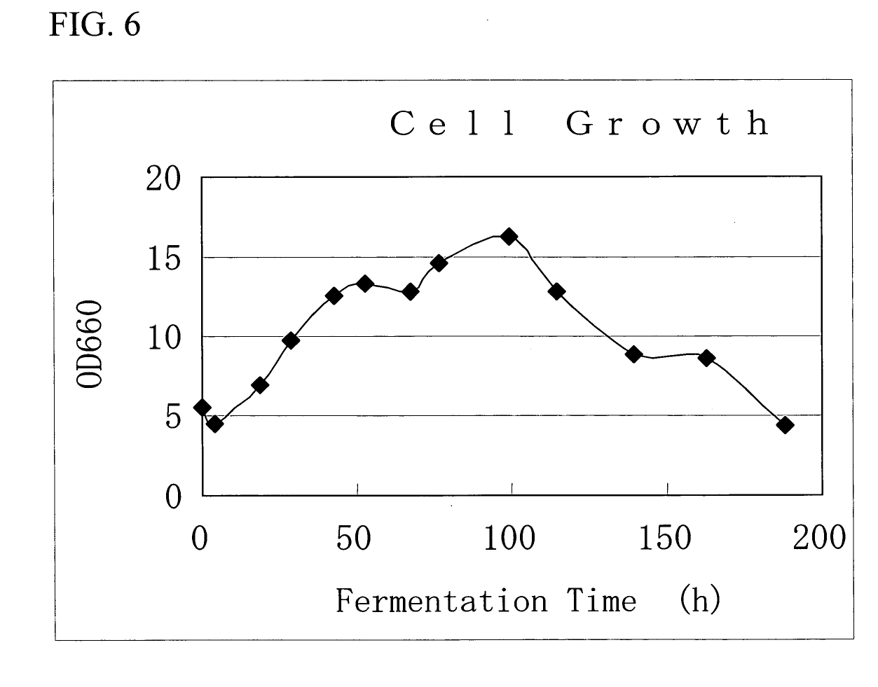

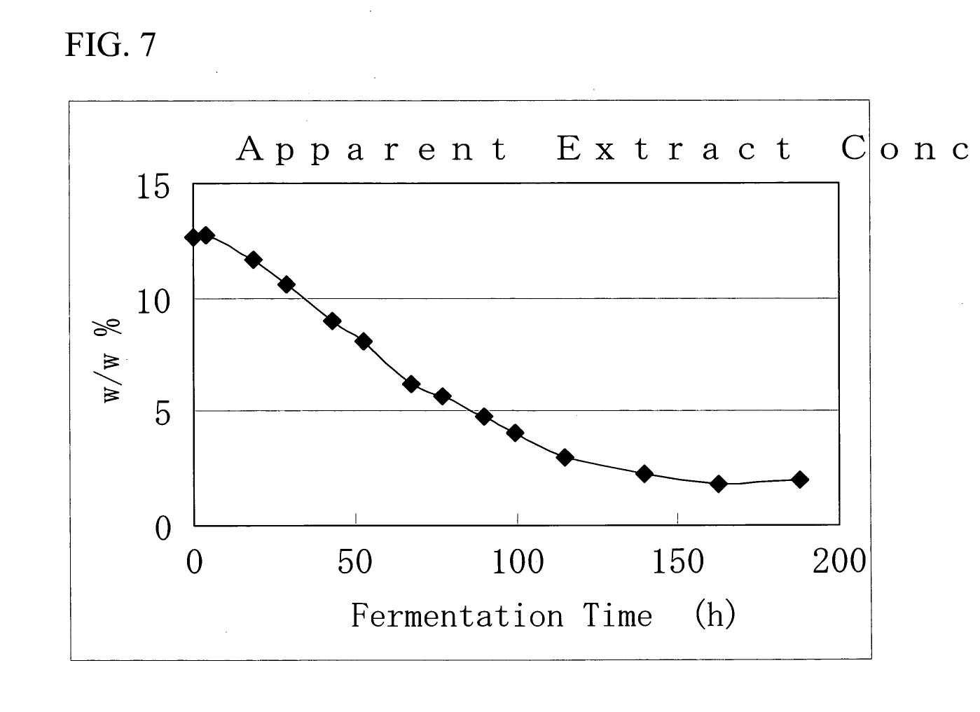

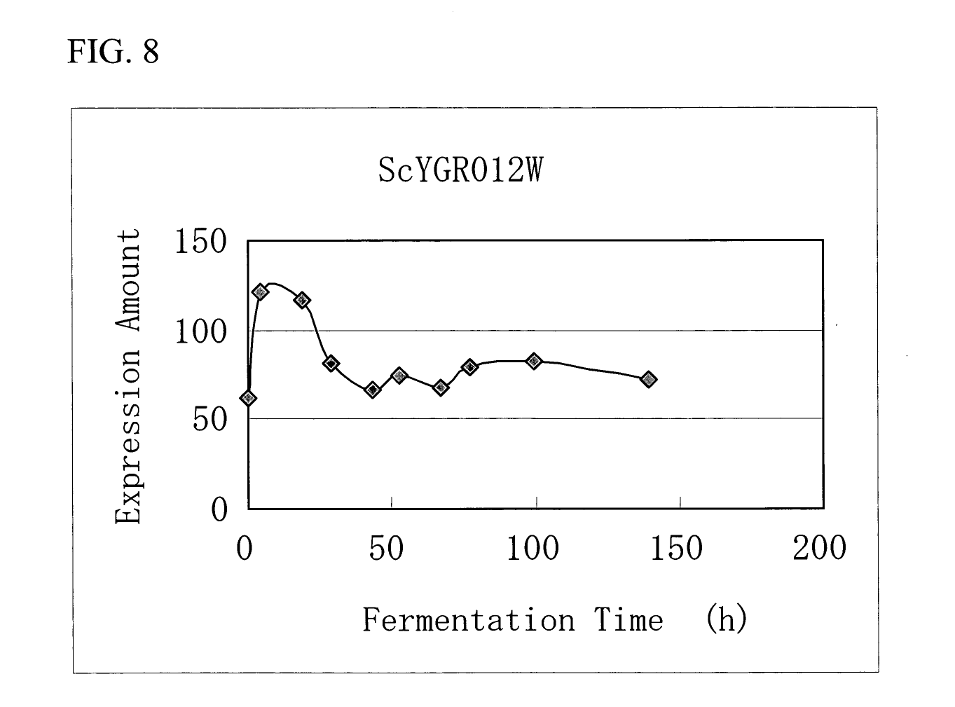

Sampling of fermented liquid was performed with time, and variation with time of yeast growth amount (FIG. 6) and apparent extract concentration (FIG. 7) was observed. Simultaneously, yeast cells were sampled to prepare mRNA, and the prepared mRNA was labeled with biotin and was hybridized to a beer yeast DNA microarray. The signal was detected using GCOS; GeneChip Operating Software 1.0 (manufactured by Affymetrix Co.). Expression pattern of ScYGR012W gene is shown in FIG. 8. As a result, it was confirmed that ScYGR012W gene was expressed in the general beer fermentation.

### Example 7

### High Expression of ScYGR012W Gene

The ScYGR012W/pCR2.1-TOPO described in Example 5 was digested using the restriction enzymes SacI and NotI so as to prepare a DNA fragment containing the entire length of the protein-encoding region. This fragment was ligated to pYCGPYNot treated with the restriction enzymes SacI and NotI, thereby constructing the ScYGR012W high expression vector ScYGR012W/pYCGPYNot. pYCGPYNot is the YCp-type yeast expression vector. The inserted gene is highly expressed by the pyruvate kinase gene PYK1 promotor. The geneticin-resistant gene G418′ is included as the selection marker in the yeast, and the ampicillin-resistant gene Ampr is included as the selection marker in Escherichia coli.

Using the high expression vector prepared by the above method, the strain Saccharomyces pasteurianus Weihenstephaner 34/70 was transformed by the method described in Japanese Patent Application Laid-open No. H7-303475. The transformant was selected in a YPD plate culture (1% yeast extract, 2% polypeptone, 2% glucose, 2% agar) containing 300 mg/L of geneticin.

### Example 8

### Analysis of Amount of Hydrogen Sulfide Produced in Test Brew of Beer

A fermentation test was carried out under the following conditions using the parent strain (W34/70 strain) and the ScYGR012W high expression strains obtained in Example 7.

| | |
|---|---|
| Wort extract concentration | 12% |
| Wort content | 1 L |
| Wort dissolved oxygen concentration | approx. 8 ppm |
| Fermentation temperature | 15° C. (fixed) |
| Yeast pitching rate | 5 g wet yeast cells/L of wort |

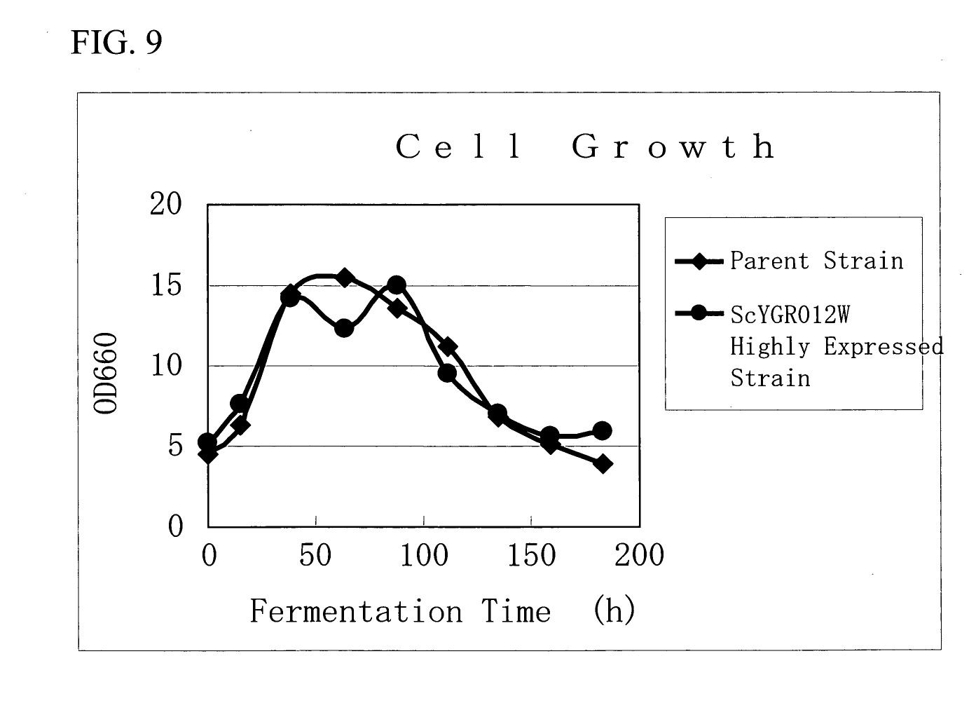

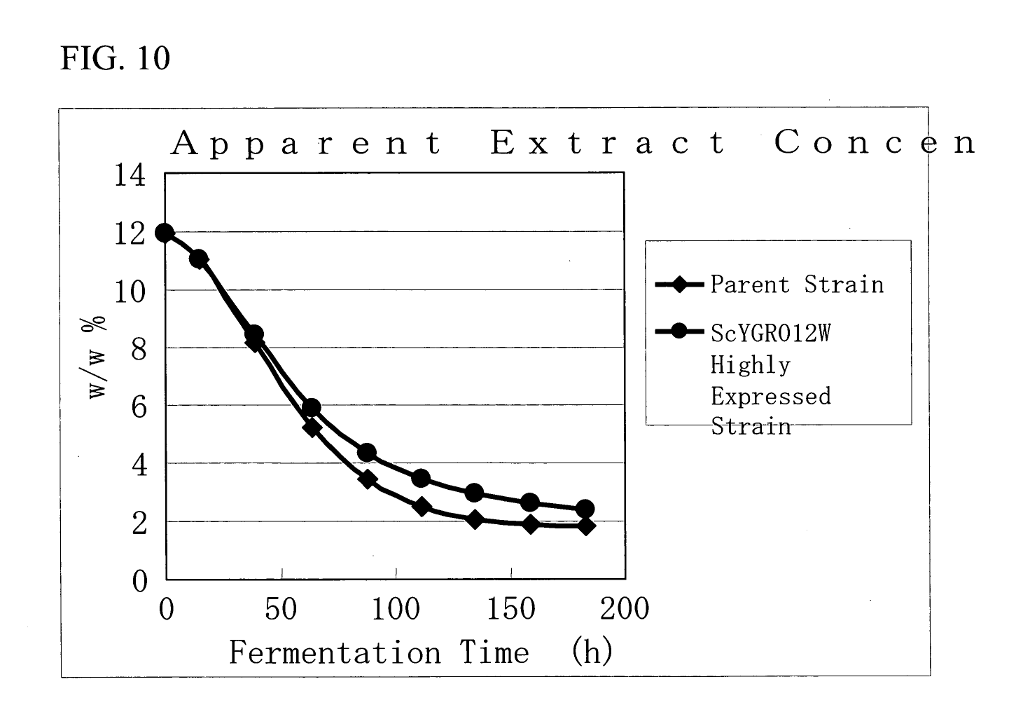

The fermentation broth was sampled over time, and the change over time in the yeast growth rate (OD660) (FIG. 9) and the amount of extract consumed were determined (FIG. 10).

Quantitative determination of the hydrogen sulfide on completion of fermentation was carried out based on the method of Takahashi et al. (Brauwissenschaft 31, 1 (1978)). First, a sample containing a known concentration of hydrogen sulfide was measured and a standard curve for hydrogen sulfide was prepared from the peak area for the hydrogen sulfide detected. The amount of hydrogen sulfide was determined from the relationship between the standard curve and the area for the hydrogen sulfide detected in measurement of the fermentation broth under the same conditions as those used for analyzing the standard sample (Table 2).

TABLE 2
Amount of hydrogen sulfide in fermentation
broth at completion of fermentation.

| | Parent strain | ScYGR012W |
|---|---|---|
| | (W34/70 strain) | highly expressed strain |
| H₂S (ppb) | 22.1 | 2.6 |

The amount of hydrogen sulfide that had been produced on completion of fermentation was 22.1 ppb for the parent strain, whereas whereas it was 2.6 ppb for the ScYGR012W highly expressed strains as described in Table 2. It was clear from these results that the amount of hydrogen sulfide production was reduced about 88% by high expression of the ScYGR012W gene.

### INDUSTRIAL APPLICABILITY

The inventive method of producing alcoholic beverages, by holding to a low level the concentration of hydrogen sulfide in beer fermentation and the finished product, can be used to produce alcoholic beverages having an excellent flavor. According to the method for producing alcoholic beverages by using a yeast transformed with a cysteine synthase (The method for producing alcoholic beverages of the present invention), hydrogen sulfide is consumed quickly by the cysteine synthase. Accordingly, concentration of hydrogen sulfide can be lowered in beer fermentation and finished product so that alcoholic beverages with superior flavor can be produced.

This application claims benefit of Japanese Patent Application Nos. 2005-240350 filed Aug. 22, 2005 and 2006-47554 filed Feb. 23, 2006, which are herein incorporated by references in their entirety for all purposes. All other references cited above are also incorporated herein in their entirety for all purposes.

1. A polynucleotide selected from the group consisting of:
(a) a polynucleotide comprising a polynucleotide consisting of the nucleotide sequence of SEQ ID NO: 1;
(b) a polynucleotide comprising a polynucleotide encoding a protein consisting of the amino acid sequence of SEQ ID NO: 2;
(c) a polynucleotide comprising a polynucleotide encoding a protein consisting of the amino acid sequence of SEQ ID NO: 2 with one or more amino acids thereof being deleted, substituted, inserted and/or added, and having a cysteine synthase activity;
(d) a polynucleotide comprising a polynucleotide encoding a protein having an amino acid sequence having 60% or higher identity with the amino acid sequence of SEQ ID NO: 2, and having a cysteine synthase activity;
(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having a cysteine synthase activity; and
(f) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO: 2 under stringent conditions, and which encodes a protein having a cysteine synthase activity.

2. The polynucleotide of claim 1 selected from the group consisting of:
(a) a polynucleotide encoding a protein consisting of the amino acid sequence of SEQ ID NO: 2, or encoding an amino acid sequence of SEQ ID NO: 2 wherein 1 to 10 amino acids thereof is deleted, substituted, inserted, and/or added, and wherein said protein has cysteine synthase activity;
(b) polynucleotide encoding a protein having 90% or higher identity with the amino acid sequence of SEQ ID NO: 2, and having cysteine synthase activity; and
(c) a polynucleotide which hybridizes to SEQ ID NO: 1 or which hybridizes to a nucleotide sequence complementary to the nucleotide sequence of SEQ ID NO: 1 under high stringent conditions, and which encodes a protein having a cysteine synthase activity.

3. The polynucleotide of claim 1 comprising a polynucleotide consisting of SEQ ID NO: 1.

4. The polynucleotide of claim 1 comprising a polynucleotide encoding a protein consisting of SEQ ID NO: 2.

5. The polynucleotide of claim 1, wherein the polynucleotide is DNA.

6. A protein encoded by the polynucleotide of claim 1.

7. A vector comprising the polynucleotide of claim 1.

8. A vector comprising the polynucleotide selected from the group consisting of:
(a) a polynucleotide encoding a protein consisting of the amino acid sequence of SEQ ID NO: 6, or encoding an amino acid sequence of SEQ ID NO: 6 wherein 1 to 10 amino acids thereof is deleted, substituted, inserted, and/or added, and wherein said protein has a cysteine synthase activity;
(b) a polynucleotide encoding a protein having 90% or higher identity with the amino acid sequence of SEQ ID NO: 6, and having a cysteine synthase activity; and
(c) a polynucleotide which hybridizes to SEQ ID NO: 5 or which hybridizes to a nucleotide sequence complementary to the nucleotide sequence of SEQ ID NO: 5 under high stringent conditions, and which encodes a protein having a cysteine synthase activity.

9. A yeast comprising the vector of claim 7.

10. The yeast of claim 9, wherein a hydrogen sulfide-producing ability is reduced by introducing the vector of claim 7.

11. The yeast of claim 10, wherein a hydrogen sulfide-producing ability is reduced by increasing an expression level of the protein encoded by the polynucleotide.

12. A method for producing an alcoholic beverage comprising culturing the yeast of claim 9.

13. The method for producing an alcoholic beverage of claim 12, wherein the brewed alcoholic beverage is a malt beverage.

14. The method for producing an alcoholic beverage of claim 12, wherein the brewed alcoholic beverage is wine.

15. An alcoholic beverage produced by the method of claim 12.

16. A method for assessing a test yeast for its hydrogen sulfide-producing capability, comprising using a primer or a probe designed based on a nucleotide sequence of a cysteine synthase gene having the nucleotide sequence of SEQ ID NO: 1 or SEQ ID NO: 5.

17. A method for assessing a test yeast for its hydrogen sulfide-producing capability, comprising: culturing a test yeast; and measuring an expression level of a cysteine synthase gene having the nucleotide sequence of SEQ ID NO: 1 or SEQ ID NO: 5.

18. A method for selecting a yeast, comprising: culturing test yeasts; quantifying the protein according to claim 6 or measuring an expression level of a cysteine synthase gene having the nucleotide sequence of SEQ ID NO: 1 or SEQ ID NO: 5; and selecting a test yeast having said protein amount or said gene expression level according to a target capability of producing hydrogen sulfide.

19. The method for selecting a yeast according to claim 18, comprising: culturing a reference yeast and test yeasts; measuring an expression level of a cysteine synthase gene having the nucleotide sequence of SEQ ID NO: 1 or SEQ ID NO: 5 in each yeast; and selecting a test yeast having the gene expressed higher than that in the reference yeast.

20. The method for selecting a yeast according to claim 18, comprising: culturing a reference yeast and test yeasts; quantifying the protein in each yeast; and selecting a test yeast having said protein for a larger amount than that in the reference yeast.

21. A method for producing an alcoholic beverage comprising:
(a) conducting fermentation for producing an alcoholic beverage using the yeast according to claim 9, or a yeast selected by a method for selecting a yeast, comprising: culturing test yeasts; quantifying the protein encoded by a polynucleotide, or measuring an expression level of a cysteine synthase gene having the nucleotide sequence of SEQ ID NO: 1 or SEQ ID NO: 5; and selecting a test yeast having said protein amount or said gene expression level according to a target capability of producing hydrogen sulfide; and wherein said polynucleotide is selected from the group consisting of:
(i) a polynucleotide comprising a polynucleotide consisting of the nucleotide sequence of SEQ ID NO: 1;
(ii) a polynucleotide comprising a polynucleotide encoding a protein consisting of the amino acid sequence of SEQ ID NO: 2;
(iii) a polynucleotide comprising a polynucleotide encoding a protein consisting of the amino acid sequence of SEQ ID NO: 2 with one or more amino acids thereof being deleted, substituted, inserted and/or added, and having a cysteine synthase activity;
(iv) a polynucleotide comprising a polynucleotide encoding a protein having an amino acid sequence having 60% or higher identity with the amino acid sequence of SEQ ID NO: 2, and having a cysteine synthase activity;
(v) a polynucleotide comprising a polynucleotide which hybridizes to a polynucleotide consisting of a nucleotide sequence complementary to the nucleotide sequence of SEQ ID NO: 1 under stringent conditions, and which encodes a protein having a cysteine synthase activity; and
(vi) a polynucleotide comprising a polynucleotide which hybridizes to a polynucleotide consisting of a nucleotide sequence complementary to the nucleotide sequence of the polynucleotide encoding the protein of the amino acid sequence of SEQ ID NO: 2 under stringent conditions, and which encodes a protein having a cysteine synthase activity; and
(b) adjusting the production amount of hydrogen sulfide.


SEQUENCE LISTING (8 sequences)

SEQ ID NO: 1 - DNA, 1188 bp, Yeast
atgtccagttcacgagataagaacgctttgtatttgccttggaacgaatgcatttccatcgcttccgtcctaatcggcgcatatgcatcgtataaatattataaactatgcaaggcgcaggatataccacgtccaaaagatggtgtggagcagctaataggaaacactcccctggttcagattagatccctttccaaggccaccggagttaacatttatgcaaaattagagttatgtaacccagcaggaagtgctaaagatagagtagcattaaacataataaagaccgcagaggaacgaggtgagctaatcagaggcgaacctggttgggtgttcgaaggaacaagcggttcaacaggcatttctatagccatggtctgtaatgcactaggatatagggcacacatttctttacccgacgacacatcgttagaaaagttggcattactagaaagtctcggtgctacagtcaataaggttaagcctgccagtattgtcgatccaaatcaatatgttaatgcagctaagaaagcatgcgatgatttgagtaaggaaggtaatggagtaaaggcagtttttgctgaccaatttgaaaatgaagccaattggagagtacactatcaaaccacgggtccagaaattgcccatcaaatggaaggaaaaattgatgcgtttattgctggctgtggtaccggtggaacgataactggagtggccaaatatctaaaggaaagcgcaaaaattccgtgccatgtcgttcttgcagatccacaaggttcaggcttttataataggataaactacggcgtgatgtatgactatgtagaaaaagaaggtacaagaaggaggcatcaggtggatactattgtagaaggtatcgggctaaacaggattactcaaaatttccacatgggcgaagagtttatcgatgaatcgatacgtgtcaatgacactcaagctatcagaatggcaaaatacctatcagtaaacgatgggttattcgtaggaagtagtacagctataaacgcagtagctgctgttcaaatcgcgaaaagactccctcctggtgccaacattgtaattattgcatgcgacagcggatcaagacatttaagtaaattctggaaagaagctaaacaaatagagtatgacatatcgttaaaggagataacaggtggcattcgatga

SEQ ID NO: 2 - protein, 395 aa, Yeast
MSSSRDKNALYLPWNECISIASVLIGAYASYKYYKLCKAQDIPRPKDGVEQLIGNTPLVQIRSLSKATGVNIYAKLELCNPAGSAKDRVALNIIKTAEERGELIRGEPGWVFEGTSGSTGISIAMVCNALGYRAHISLPDDTSLEKLALLESLGATVNKVKPASIVDPNQYVNAAKKACDDLSKEGNGVKAVFADQFENEANWRVHYQTTGPEIAHQMEGKIDAFIAGCGTGGTITGVAKYLKESAKIPCHVVLADPQGSGFYNRINYGVMYDYVEKEGTRRRHQVDTIVEGIGLNRITQNFHMGEEFIDESIRVNDTQAIRMAKYLSVNDGLFVGSSTAINAVAAVQIAKRLPPGANIVIIACDSGSRHLSKFWKEAKQIEYDISLKEITGGIR

SEQ ID NO: 3 - DNA, 40 bp, Artificial
  Primer
gagctcatagcggccatgtccagttcacgagataagaacg

SEQ ID NO: 4 - DNA, 42 bp, Artificial
  Primer
ggatcctatgcggccgcggaaaaactaacaaggaattcagaa

SEQ ID NO: 5 - DNA, 1182 bp, Yeast
atgtcctgttctcaaaataagacttctgtcagtctggcctggcgtgaatgcatatccatcgcttccgtcctaattggcgcgtatgcatcatacagatactataagttgttcaaaacacgcgatattccacgtccaaaagaaggcattgaagaactcataggaaacactcctctggttaagatcagatctcttaccaaggctactggagttaacatttacgctaagttggagttatgcaacccagcaggaagtgctaaggatagagtggctttaaacatcataaagacggctgaggaattaggcgagctcgtcagaggtgaacctggctgggtatttgaaggaacaagtgggtcgactggtatctctatagcagtggtctgtaatgcgttgggttatagggcacacatttctttgcccgatgacacatcgctagaaaaattggcattactagaaagcctcggtgctactgttaataaggttaagcccgctagtattgtcgatccaaaccaatacgtaaatgccgccaaaaaggcatgtaatgaactaaagaagagtggtaatggaataagagcagtttttgccgatcagttcgaaaacgaagccaactggaaagtacattaccagaccacaggcccagaaattgcccatcaaactaagggaaatattgacgcattcattgctggttgcggtactggtggcaccataactggtgtggcaaaattcttaaaggaaagagcaaagataccttgccatgtcgttctagcggatccacaaggatctggcttttataatagagtcaactatggcgtgatgtatgactatgtagagaaggaaggtactagaagaaggcatcaagttgacactatcgtagaagggattgggttaaacagaatcactcataattttcatatgggcgaaaaatttattgatgaatcaatacgagtcaacgataatcaagccattagaatggcaaaatacctatcggtaaacgacgggctattcgtggggagtagtacagctattaatgcggtagccgctattcaggttgcaaaaacgcttcctcacggctcaaacattgtgattattgcatgcgacagcggttcaagacacctaagtaaattctggaaagaagctaaagaaatcgatcacgacgtatcattagaagaagtaataaatatctaa

SEQ ID NO: 6 - protein, 393 aa, Yeast
MSCSQNKTSVSLAWRECISIASVLIGAYASYRYYKLFKTRDIPRPKEGIEELIGNTPLVKIRSLTKATGVNIYAKLELCNPAGSAKDRVALNIIKTAEELGELVRGEPGWVFEGTSGSTGISIAVVCNALGYRAHISLPDDTSLEKLALLESLGATVNKVKPASIVDPNQYVNAAKKACNELKKSGNGIRAVFADQFENEANWKVHYQTTGPEIAHQTKGNIDAFIAGCGTGGTITGVAKFLKERAKIPCHVVLADPQGSGFYNRVNYGVMYDYVEKEGTRRRHQVDTIVEGIGLNRITHNFHMGEKFIDESIRVNDNQAIRMAKYLSVNDGLFVGSSTAINAVAAIQVAKTLPHGSNIVIIACDSGSRHLSKFWKEAKEIDHDVSLEEVINI

SEQ ID NO: 7 - DNA, 40 bp, Artificial
  Primer
gagctcatagcggccatgtcctgttctcaaaataagactt

SEQ ID NO: 8 - DNA, 42 bp, Artificial
  Primer
ggatcctatgcggccgcccacggaaagtagcaaaaaatgtag